(19) United States
(12) Patent Application Publication
(10) Pub. No.: US 2025/0144314 A1
(43) Pub. Date: May 8, 2025

(54) VITAMIN K1 INJECTION DEVICE KIT

(71) Applicant: ORBICULAR PHARMACEUTICAL TECHNOLOGIES PRIVATE LIMITED, Hyderabad (IN)

(72) Inventors: Hiren PATEL, Hyderabad (IN); Satish Kumar SIKHAKOLLI VENKATA, Hyderabad (IN); Sapnil Kumar PATEL, Hyderabad (IN)

(21) Appl. No.: 18/730,092

(22) PCT Filed: Sep. 12, 2022
(86) PCT No.: PCT/IB2022/058565

§ 371 Date: Jul. 18, 2024

(30) Foreign Application Priority Data
Jan. 20, 2022 (IN) 202241003304

(51) Int. Cl. A61M 5/315; A61K 9/00

(52) U.S. Cl. CPC A61M 5/31595; A61K 9/0029

(57) ABSTRACT

The present invention relates to a simple, convenient, user-friendly, ready to administer pre-filled syringe injection device, for delivery of medication composition used for the prophylaxis and treatment of vitamin K deficiency bleeding in the neonates and other age groups.

### FIELD OF THE INVENTION

This invention relates to a simple, convenient, user-friendly, ready to administer pre-filled syringe injection device, for delivery of medication product used for the prophylaxis and treatment of vitamin K deficiency bleeding in the neonates and other age groups.

### BACKGROUND OF THE INVENTION

Vitamin K (Phytonadione) is used to prevent bleeding in people with blood clotting problems or too little vitamin K in the body. Phytonadione is in a class of important medications called vitamins. It works by providing vitamin K that is needed for blood to clot normally in the body. It helps to make various proteins that are needed for blood clotting and the building of bones. It functions as a cofactor required for the activity of vitamin K-dependent proteins, which include factors II (prothrombin), VII, IX, and X, in addition to protein C and protein S. Prothrombin is a vitamin K-dependent protein directly involved with blood clotting. Osteocalcin is another protein that requires vitamin K to produce healthy bone tissue.

Vitamin K1 is a fat-soluble vitamin that is present in leafy green vegetables. It is essential for normal coagulation. In vitamin K deficiency, phytonadione therapy replenishes stores. Vitamin K is absorbed in the intestinal tract similarly to the way that most dietary lipids are absorbed by uptake into micelles composed of bile salts. They are absorbed into intestinal enterocytes into the lymphatic system and reach venous circulation by way of the thoracic duct. Estimates are that over 60% of absorbed vitamin K will be excreted from the body. Phytonadione is metabolized in the liver and then excreted in the bile and urine.

Vitamin K is approved by major regulatory agencies and marketed in several countries and available as Phytonadione injection or Vitamin K injection and is indicated for treatment of Hypoprothrombinemia due to Vitamin K deficiency or interference, and prophylaxis and treatment of Vitamin K-deficiency bleeding in neonates.

The USFDA approved Phytonadione Injectable Emulsion, USP, 1 mg/0.5 mL on Mar. 1, 1976 to International Medication Systems (IMS) for treatment of Hypoprothrombinemia due to Vitamin K Deficiency or interference and prophylaxis and treatment of Vitamin K-deficiency bleeding in neonates. Phytonadione Injectable Emulsion, USP is available in 1 mg (1 mg/0.5 mL) single-dose vials and a SAF-T-Jet® vial injector. IMS Phytonadione Injection drug product contains 2 mg phytonadione, 20 mg polysorbate 80, 20.8 mg propylene glycol, 0.34 mg sodium acetate, and 0.00004 mL glacial acetic acid in water for injection. Additional glacial acetic acid or sodium acetate anhydrous may have been added to adjust pH to meet USP limits of 3.5 to 7.0. The air above the liquid in the individual containers has been displaced by flushing with nitrogen during the filling operation. However other Phytonadione injectable products with different compositions are also commercially available in the market.

IMS phytonadione injection is available and supplied as Phytonadione Injectable Emulsion USP, 1 mg/0.5 mL injector is a disposable, manually operated, supplied in unit use packages containing one single-dose vial and a SAF-T-Jet® vial injector, 27 G×½″ needle which needs to be assembled before administration. At the time of injection, the single-dose vial needs to be aligned and assembled to vial injector, and the drug is injected by threading the vial into injector with three half turns. Further, assembling SAF-T-Jet® vial injector has several steps which has to be done by skilled clinician or nurse at the healthcare setup.

SAF-T-JET vial is available as a 2-piece device consisting of single dose vial or cartridge prefilled with the medication and SAF-T-JET vial injector attached with 27 G×½″ safety needle, which has to be further assembled for ready administration to the patient.

Steps to be performed before administration of phytonadione injection through SAF-T-Jet® vial.
1. Remove the protective caps from vial and injector.
2. Align vial such that the injector needle is centered on the stopper.
   Thread vial into injector 3 half turns; this will allow the needle to penetrate the stopper* (*CAUTION: IMPROPER ENGAGING MAY CAUSE GLASS BREAKAGE AND SUBSEQUENT INJURY)
   DO NOT PUSH VIAL INTO INJECTOR; THIS MAY CAUSE MISALIGNMENT OF THE INJECTOR NEEDLE.
3. i. Flip the shield down
   ii. Remove Needle cover pulling straight up (DO NOT TWIST). Expel air.
4. i. After injection, push tab forward with thumb to flip shield toward needle.
   ii. With a FIRM, QUICK motion, press down against a flat surface until an audible “click” is heard. This sound indicates shield activation. Note: Audible click may not be heard on small needle sizes: visual confirmation is required.

Further to this the vial or cartridge has the dose marking such as (1 mg-0.5 mL and 0.5 mg-0.25 mL) on the label which is clearly visible before insertion to the SAF-T-Jet vial. However, once the vial or cartridge is inserted into the SAF-T-Jet vial, clear visibility of the cartridge is difficult as the SAF-T Jet vial is opaque in nature and has 2 layers within. Though the cartridge is prefilled, visibility of the dose marking is difficult to know if the dose has been completely dispensed or administered to the patient leading to dosing errors which is undesirable. Required dosing of Vitamin K has to be administered to the patients, dosing errors may lead to other major complications.

SAF-T-jet injector vial is disclosed in U.S. Pat. No. 3,739,780, wherein it discloses a medicament injector comprising a cylindrical vial.

U.S. Pat. Nos. 10,028,921 and 11,096,907 discloses stable Phytonadione compositions for parenteral administration are provided which comprise (E) isomer of phytonadione at or greater than 97% w/w as the active ingredient and is substantially free of (Z) isomer. Said compositions are stable, sterile, and particulate-free. Further, said compositions reduce or avoid allergic reactions to benzyl alcohol and polysorbate. Further, it mentions that the compositions may be administered in a non-reacting glass or non-reacting polymeric container selected from a vial container or a pre-filled syringe container.

US20190328971A1 discloses apparatus includes a housing, a medicament container and a movable member. The medicament container is configured to move within the housing between a first position and a second position in response to a force produced by an energy storage member. Dependent claim 3 claims Vitamin B12 as one of component.

SAF-T-jet device has disadvantages such as, assembling of drug vial and in the event of improper handling of SAF-T-jet device during assembling it may result in glass breakage and subsequent injuries.

Considering above steps, precautions for preparation of the SAF-T jet injection product, which is tedious, time consuming, and skilled persons are required to prepare the same. Further, in order to understand the procedure or any error during the injection preparation steps may cause unnecessary delay of medication to the patient and also the product may get damaged due to the errors in preparation. Further, there may be dose variabilities leading to further consequences if the precautions are not taken care of during the device preparation.

In view of the above complexities of preparation and administration of the medication, there is unmet need in the art to develop a simple, easy and quick delivery device to administer the medicine to the patient. Hence, the present invention provides a simple, convenient, user-friendly ready to administer delivery device. The present invention provides ease of administration of vitamin K1 injection to the patient by pre-filled syringe or auto injector injection device.

### SUMMARY OF THE INVENTION

One object of the present invention is to provide ease of administration of vitamin K1 injection to the patient.

Another object of the present invention is to provide a simple, convenient, user-friendly, ready to administer pre-filled syringe injection device, for delivery of medication product used for the prophylaxis and treatment of vitamin K deficiency bleeding in the neonates and other age groups.

Another object of the present invention is to provide a simple, convenient, user-friendly auto injector injection device, for delivery of medication product used for the prophylaxis and treatment of vitamin K deficiency bleeding in the neonates and other age groups.

In one embodiment, of the present invention is to provides ready to use, ready to administer, ready to inject, or ready to dilute composition of emulsion, colloidal solution and/or other biphasic liquid medication comprising therapeutic quantity of vitamin K1.

In one embodiment, of the present invention provides the composition of Vitamin K1 delivered through parenteral routes such as subcutaneous, intramuscular, or intravenous route of administration.

In one embodiment, the present invention provides the composition of Vitamin K1 can be injected in the range of 1 mg to 50 mg as unit dose compositions based on the requirement.

In one embodiment, the present invention provides the prefilled syringe injection device with a staked needle with rigid needle shield wherein, the medication is injected by pushing the pre-attached plunger rod.

In one embodiment, the present invention provides ready to use prefilled syringe system is accompanied with suitable safety device system.

In one embodiment, the present invention provides prefilled injection device which doesn't require pre-assembling with multiple steps before administration.

In one embodiment, the present invention injection device, provide better shield performance and/or functionality of safety needle in comparison to other devices.

In one embodiment, the present invention injection device comprises injectable composition used for the treatment of Vitamin K deficiency in emergency use.

In one embodiment, the present invention provides prefilled syringe injection device comprises injectable composition of Vitamin K1 is used for the treatment of Vitamin K deficiency related disease and disorders.

In one embodiment, the present invention provides prefilled syringe injection device comprises injectable composition of Vitamin K1 is used for the treatment of Vitamin K sensitive disease and disorders.

### DETAILED DESCRIPTION OF THE INVENTION

The present invention relates to a simple, convenient, user-friendly, ready to administer pre-filled syringe injection device, for delivery of medication.

The present invention relates to a simple, convenient, user-friendly, ready to administer pre-filled syringe injection device, for delivery of medication product used for the prophylaxis and treatment of vitamin K deficiency bleeding in the neonates and other age groups.

The term “Vitamin K” or “phytonadione” includes the compound, pharmaceutically acceptable salts thereof, isomers, solvates, prodrugs, complexes and hydrates, anhydrous forms thereof, and any polymorphic or amorphous forms or combinations thereof.

The term “vitamin K” as used herein includes a group of lipophilic, hydrophobic vitamins that are needed for the post-translational modification of certain proteins, mostly required for blood coagulation. Chemically, they are 2-methyl-1,4-naphthoquinone derivatives. Vitamin K1 is also known as phylloquinone or phytomenadione (also called phytonadione), and vitamin K2 is also known as menaquinone or menatetrenone. There are three synthetic forms of vitamin K, vitamins K3, K4, and K5, which are used in many areas including the pet food industry (vitamin K3) and for inhibiting fungal growth (vitamin K5).

The term “pharmaceutically acceptable salts” includes pharmaceutically acceptable salts, solvates, hydrates, anhydrates, enantiomers, esters, isomers, polymorphs, tautomers, complexes and thereof.

The term “ready-to-use” or “ready to administer” or “ready to inject” or “ready to dilute” refers to a liquid composition for parenteral administration.

The terms “composition” and “formulation” refer to preparations comprising Vitamin K or Phytonadione or phytomenadione or Vitamin K1 or phylloquinone or pharmaceutically acceptable salts thereof with pharmaceutically acceptable solvent or carriers or excipients; in a form suitable for administration to a mammal.

The terms “injectable composition”, or “injection composition” or “injectable formulation” or “injection formulation” refer to preparations comprising Vitamin K or Phytonadione or phytomenadione or Vitamin K1 or phylloquinone or pharmaceutically acceptable salts thereof; in a form suitable for administration to a mammal.

The term “Therapeutically effective amount” or “therapeutic quantity” means an amount of a compound that, when administered to a subject for treatment or prophylaxis of disease or disorder or amount of a compound that is effective in therapy, or an amount sufficient to provide a therapeutic effect. The “therapeutically effective amount” or “therapeutic quantity” can vary depending on the compound, the disease and its severity, and the age, weight, etc., of the subject to be treated.

The term “substantially dissolved” relates to 0.1% to 99% of total Vitamin K or Phytonadione or phytomenadione or Vitamin K1 or phylloquinone or pharmaceutically acceptable salts thereof is dissolved in the compositions.

The term “stable” or “stability” as used herein includes both physical and chemical stability. Stability parameters include but not limited to potency, stable pH value and other important physico-chemical parameters.

The term “physical stability” refers to compositions free from particles and that do not significantly change during storage.

The term “chemical stability” relates to a limited formation of impurities, limited decrease in potency and the like.

Vitamin K may exist as a mixture of two isomers, known as (E) and (Z) isomers. The precise ratio of these isomers in a given drug product composition is unknown. Some commercial preparations may contain up to 20% of the cis (Z) isomer. See for example, Budavari, S. (ed.). The Merck Index—Encyclopedia of Chemicals, Drugs and Biologicals. Rahway, N.J.: Merck and Co., Inc., 1989., p. 1580. Commercially available phylloquinone (Phytonadione) is prepared synthetically and may contain not only 2′,3′-trans-phylloquinone (not less than 75%), but also 2′,3′-cis-phylloquinone (up to 21% as per USP XXXII).

Existing Phytonadione or Vitamin K injections are supplied and commercially available as ampoules and SAF-T-jet injection vials. Packaging material plays a very important role for finished pharmaceutical compositions. In packaging materials there are primary packaging, secondary packaging materials. Primary Packaging material plays an important role while packaging the finished pharmaceutical composition as the composition is in direct contact with the primary packaging material.

Primary packaging generally includes ampoules, vials, cartridges, bottles, bags, and the like. Further to close or to seal the primary containers, elastomers are widely used in primary parenteral packaging as stoppers for vials, plungers and tip caps for pre-fillable syringes, plungers and seals for cartridges and ports for plastic bags. Hence, choosing the primary packaging is of prime importance. As the phytonadione injection is supplied in ampoules, use of ampoules for this injection has several disadvantages.

Dose wastage while withdrawing medication from the ampoule as it must be scored and broken, indirectly leading to dose variation if there is any spillage from ampoule. Further while withdrawing the composition in a syringe from ampoule there may be some left over of the solution again leading to dose variation. Further, there may be stability issue due to head space in the ampoule leading to degradation of the composition. While breaking the ampoule, it is likely that small glass pieces may enter in the product and may go unnoticed. This may be very serious issue, as the proposed formulation is intended for neonatal age population. Hence, there are several disadvantages of the composition when supplied in ampoules.

Similarly, SAF-T-jet injector vial has several disadvantages as mentioned in the background of this invention.

Therefore, considering these issues the present invention provides a simple, convenient, user-friendly pre-filled syringe injection device, for delivery of medication to the patients which overcomes the above issues or complications.

Further, the present invention provides a simple, convenient, user-friendly auto injector device, for delivery of medication to the patients which overcomes the above issues or complications.

The commercially available Vitamin K injectable ampoules may have stability issues or less stable due to the head space oxygen. Though nitrogen gas is purged after the ampoule filling there may be some nascent oxygen leading degradation of composition.

The present invention provides injection device to overcome the stability issues which have been existing in the ampoules due to the head space oxygen.

The present invention solves the stability issues by providing injection devices such as prefilled syringe, autoinjectors where these issues are taken care by the proper filling and minimal head space and hence stability is noticed to satisfactory levels.

Phytonadione injection is also supplied in ampoules, wherein the use of ampoules has its own drawbacks, such as before administration the health care worker or clinician has to keep the syringe, ampoule ready. Further to break the ampoule a clean snap should be used, while breaking if too much force is applied it can cause the ampoule to shatter, if the ampoule shatters, discard it and use a new ampoule, using the new ampoules, hence leading to loss of the ampoule. Another drawback of the ampoule is while cutting the ampoule small glass pieces may also drop inside the ampoule, which may go unnoticed and if not observed it may be difficult to identify as vitamin K injection is biphasic liquid or colloidal solution. Hence this minute glass pieces may be administered to the patients or sometimes, they may choke the needles leading to improper withdrawal of the injection and final wastage of the ampoules.

During emergency conditions, pre-filled syringe is very useful and important for immediate and quick action to the patients. As Vitamin K or phytonadione injection is used to cure the Vitamin K related deficiencies, which are linked to the blood clotting factors, hence these are the most critical injection that acts in life saving indirectly. Hence, injectable pre-filled syringe device is very useful in these types of situations.

During emergency conditions, auto injectors are very useful and important for immediate and quick action to the patients. As Vitamin K or phytonadione injection is used to cure the Vitamin K related deficiencies, which are linked to the blood clotting factors, hence these are the most critical injection that acts in life saving indirectly. Hence, prefilled syringe or auto injector device is very useful in these types of situations.

In one embodiment, the present invention provides prefilled injection device are composed of the barrel, piston, plunger rod, needle and tip cap or needle shield, and includes a variety of modification options for each of these components.

In one embodiment, the present invention provides prefilled injection device with improved safety of needles comprises introduction of retractable needles. A syringe equipped with this option appears with the needle extended from the syringe prior to use and the needle retracts into the syringe when the plunger is completely depressed.

In one embodiment, the present invention provides prefilled injection device with staked or fixed needle.

In one embodiment, the present invention provides prefilled injection device which is operated manually and supplied in unit use packages containing one prefilled syringe set affixed with a 27 G×½″ needle (staked needle) with rigid needle shield cap and safety needle shielding system.

In one embodiment, the present invention injector device has graduations or markings is pre-printed on the syringe barrel of the container making it visible to the clinician or health care practitioner.

In an embodiment, assembling of drug vial is not required for present invention device, and in the event of improper handling of SAF-T-jet device during assembling may result in glass breakage and subsequent injuries.

In one embodiment, the pre-filled syringes are filled with therapeutically required composition and the plunger rod is previously inserted.

In one embodiment, the present invention provides injection device having safety needle shield system, has an advanced feature that the shield shall be activated automatically by firmly pushing the plunger soon after the dose is administered.

In one embodiment, the present invention injector device is disposable, manually operated, supplied in unit use packages containing one prefilled syringe set affixed with staked needle with rigid needle shield cap and safety needle shielding system.

Another aspect, the present invention is not limited solely to such a prefilled syringe device in foregoing description. A person skilled in the art understands that various modifications to the described prefilled syringe device may be implemented within the scope of the present disclosure.

In one embodiment, the pre-filled syringe device as such can be discarded easily since the device is equipped with the safety device.

In one embodiment, the present invention injection device minimizes the handling errors like improperly attached needle leads to leaking of the therapeutic composition, thereby leading to less dose or dose inaccuracy.

In another embodiment, the present invention injection device reduces the device handling/assembling time and reduces the risks by eliminating the additional steps before and after administration of the injection.

In another embodiment, present invention injection device enhances user convenience, reduces risk of contamination and is familiar to the end users.

In another embodiment, present invention provides staked needle syringe device which provides the safety to the patient by maintaining the integrity of the drug product.

In one embodiment, the compositions are provided to a subject in need thereof with a composition in a container that is amenable to deliver the ready to use composition. Said container is provided in 1 ml, 2 ml, 5 ml, 10 ml, glass or plastic vial. Alternatively, a ready-to-administer composition may be provided in a pre-filled syringe. Such pre-filled syringes are able to deliver drug solution volumes from about 0.1 ml to about 10 ml. For example, about 0.1 ml, 0.2 ml, 0.25 ml, 0.3 ml, 0.4 ml, 0.5 ml, 0.75 ml, 1.0 ml, 1.25 ml, 1.5 ml, 2.0 ml, 3.0 ml, 5.0 ml, 10 ml, of drug solution volume can be delivered to patients. In all cases, the containers of said vials or pre-filled syringes may be made of type-1 glass, or non-reacting polymeric material such as polypropylene or polyethylene or a mixture thereof. Several non-reacting containers are known in the art. The non-reacting containers described herein include not only the vial or pre-filled syringe that makes up the bulk of the container structure, but also any other part of the container that comes in contact with the drug solution, such as stoppers, plungers, etc. These also are made of type-1 glass or non-reacting polymeric materials.

In one aspect the injection composition is administered intramuscularly. In another aspect, the injection compositions are administered subcutaneously or intravenously.

In some embodiments, the devices can also prevent device tampering by a user and/or multiple uses of the same syringe device. In some embodiments, the syringe devices described herein can be used in a critical situation for delivery of an emergency and/or time sensitive pharmaceutical agent in response to a drug or substance overdose. The syringe devices can include a syringe that includes a dose of at least one pharmaceutical agent and can deliver a therapeutic dose of the pharmaceutical agent that can be less than the amount of pharmaceutical agent in the syringe.

The term “Vitamin K sensitive diseases or disorders” or “Vitamin K deficiency sensitive diseases or disorders” or “Vitamin K deficiency related disease and disorders” as used herein includes but not limited to preventing and/or treating haemorrhagic disease of the newborn and/adults, blood coagulation disorders of the newborn and/adults, treatment of a haemorrhagic disease, treatment of Hypoprothrombinemia due to Vitamin K deficiency or Interference, coagulation disorders which are due to faulty formation of factors II, VII, IX and X, anticoagulant-induced hypoprothrombinemia caused by coumarin or indanedione derivatives, hypoprothrombinemia due to antibacterial therapy, hypoprothrombinemia secondary to factors limiting absorption or synthesis of vitamin K, e.g., obstructive jaundice, biliary fistula, sprue, ulcerative colitis, celiac disease, intestinal resection, cystic fibrosis of the pancreas, and regional enteritis, other drug-induced hypoprothrombinemia where it is definitely shown that the result is due to interference with Vitamin K metabolism, e.g., salicylates, prophylaxis and treatment of Vitamin K-deficiency bleeding in neonates and/adults.

In one embodiment, the present invention provides prefilled syringe injection device comprises injectable composition of Vitamin K1 is used for the treatment of Vitamin K sensitive diseases and disorders.

In one embodiment, the present invention provides prefilled syringe injection device comprises injectable composition of Vitamin K1 is used for the treatment of Vitamin K deficiency related diseases and disorders.

To further illustrate the invention, the following example is provided. It is to be understood that these examples are provided for illustrative purposes and are not to be construed as limiting the scope of the present invention in any manner whatsoever.

### Example 1

TABLE 01
Vitamin K1 Composition

| S. No. | Material Description | Qty/mL | | Qty./unit | |
|---|---|---|---|---|---|
| 1 | Vitamin K1 | 2.00 | mg | 1.00 | mg |
| 2 | Polysorbate 80 | 20 | mg | 10.0 | mg |
| 3 | Propylene glycol | 20.8 | mg | 10.4 | mg |
| 4 | Sodium acetate anhydrous | 0.34 | mg | 0.17 | mg |
| 5 | Glacial acetic acid | 0.00004 | mL | 0.00002 | mL |
| 6 | Water for Injection | q.s to 1 | mL | q.s to 0.5 | mL |

The stability studies were performed for composition mentioned in Table 1 for two different batches (Batch 1 & Batch 2).

The 6-month stability studies were performed for Batch 1 of Vitamin K1 composition mentioned in Table 1. The stability of the Vitamin K1 composition was monitored at different stability conditions such as 25° C./60% RH and 40° C./75% RH for 6 months and results seem to be satisfactory.

The stability study results for batch 1 are summarized in table 2 below:

TABLE 02
Experimental details (Batch 1)

| | Time points | | |
|---|---|---|---|
| | Initial - 25° | 6 M - 25° | 6 M - 40° |
| Parameters | C./60% RH | C./60% RH | C./75% RH |

Physical parametres

| | | | |
|---|---|---|---|
| Description | CYS | CYS | CYS |
| pH | 5.7 | 5.8 | 5.9 |
| Assay | 103.8% | 103.5% | 92% |
| Total | 0.74% | 2.24% | 5.14% |
| impurities | | | |

#CYS—Clear Yellow Solution,
RH—Relative humidity

The long-term stability studies were performed for Batch 2 of Vitamin K1 composition mentioned in Table 1. The stability of the Vitamin K1 composition was monitored for 3 months at 25° C./60% RH, 6 months at 25° C./60% RH and 12 months at 25° C./60% RH and results seems to be satisfactory.

The stability study results for batch 2 are summarized in table 3 below:

TABLE 03
Experimental details (Batch 2)

| | Time Points | | | |
|---|---|---|---|---|
| | | 3 M - 25° | 6 M - 25° | 12 M - 25° |
| Parameters | Initial | C./60% RH | C./60% RH | C./60% RH |
| Description | CYS | CYS | CYS | CYS |
| pH | 5.6 | 5.4 | 5.5 | 5.6 |
| Assay | 105.7% | 104.9% | 105.0% | 99.0% |
| Total | 0.77% | 1.62% | 2.53% | 4.45% |
| Impurities | | | | |

CYS—Clear Yellow Solution;
RH: Relative Humidity

We claim:

1. A ready to administer pre-filled syringe injection device, comprising: composition comprises therapeutically effective amount of vitamin K; wherein said device provides ease of administration in patients for the prophylaxis and treatment of vitamin K deficiency bleeding,
wherein said device is simple, convenient and user-friendly.

2. The pre-filled syringe injection device as claimed in claim 1, comprises ready to use, ready to administer, ready to inject, or ready to dilute composition of emulsion, colloidal solution and/or other biphasic liquid medication composition comprising therapeutic quantity of vitamin K1.

3. The pre-filled syringe injection device as claimed in claim 1, are used for administration of Vitamin K1 injectable composition parenterally such as subcutaneous, intramuscular, or intravenous injection.

4. The pre-filled syringe injection device as claimed in claim 1, provides ease of administration in patients like neonates and other age groups.

5. The pre-filled syringe injection device as claimed in claim 1, comprises the medication Vitamin K1 in the range of 1 mg to 50 mg as unit dose or multidose compositions.

6. The pre-filled syringe injection device as claimed in claim 1, is provided with a staked needle having rigid needle shield; wherein the medication is injected by pushing the pre-attached plunger rod.

7. The injection device as claimed in claim 1, is ready to use prefilled syringe system accompanied with suitable safety device system.

8. The prefilled syringe injection device as claimed in claim 1, is provides ease of administration without pre-assembling of device with multiple steps before administration.

9. The prefilled syringe injection device as claimed in claim 1, used to treat Vitamin K deficiency related diseases and disorders.
